(19) United States
(12) Patent Application Publication
(10) Pub. No.: US 2025/0375404 A1
(43) Pub. Date: Dec. 11, 2025

(54) AN ORODISPERSIBLE PHARMACEUTICAL COMPOSITION OF BACLOFEN AND ITS PROCESS OF PREPARATION

(71) Applicant: NOVUMGEN LIMITED, London (GB)

(72) Inventor: Kamleshkumar PATEL, London (GB)

(21) Appl. No.: 18/878,567

(22) PCT Filed: Jun. 21, 2023
(86) PCT No.: PCT/GB2023/051618

§ 371 Date: Dec. 23, 2024

(30) Foreign Application Priority Data
Jun. 24, 2022 (GB) 2209281.1

(51) Int. Cl. A61K 31/197; A61K 9/00; A61K 47/12; A61K 47/18; A61K 47/26; A61K 47/38

(52) U.S. Cl. CPC A61K 31/197; A61K 9/0056; A61K 47/12; A61K 47/183; A61K 47/26; A61K 47/38

(57) ABSTRACT

The present invention relates to orodispersible pharmaceutical composition suitable for oral administration comprising Baclofen with at least one disintegrant and at least one diluent. In the preferred embodiment Sodium starch glycolate is used as disintegrant in the range about in the range from about 1% w/w to about 10% w/w, preferably in the range from about 2% w/w to about 7.5% w/w. The present invention also relates to process for preparing the said solid pharmaceutical composition. The wet granulation process is used for the manufacturing process.

### FIELD OF THE INVENTION

The present invention relates to pharmaceutical composition of Baclofen. The present invention more particularly relates to orodispersible pharmaceutical composition of Baclofen for Oral administration. The present invention also relates to process of the preparation of the same.

### BACKGROUND OF THE INVENTION

Baclofen was disclosed in the NL 6407755. Baclofen is a skeletal muscle relaxant and Antispastic/Antispasmodic agent. Baclofen is a structural analog of the inhibitory neurotransmitter gamma-aminobutyric acid (GABA), and may exert its effects by stimulation of the GABAB receptor subtype.

Baclofen is clinically found to be effective in the treatment of muscle spasms caused by certain conditions (such as multiple sclerosis, spinal cord injury/disease).

The commercially marketed products of Baclofen in tablet form are available in three dosage strengths: 5 mg, 10 mg and 20 mg for oral administration. The marketed product tablet form is used in the treatment of spasticity resulting from multiple sclerosis, particularly for the relief of flexor spasms and concomitant pain, clonus, and muscular rigidity. It is also being of some value in patients with spinal cord injuries and other spinal cord diseases.

Few more formulations of Baclofen are available in the market as solution, suspension, granules or as an injectable form.

Intrathecal (baclofen injection) has been developed for chronic intrathecal infusion for the management of severe spasticity. Baclofen is commercially available for intrathecal injection as a 0.05 mg/mL solution, a 0.5 mg/mL solution or a 2 mg/mL solution having a pH of 5 to 7 in a formulation containing sodium chloride and water.

The currently available solid dosage form of Baclofen has relatively lengthy onset times. Further, it is also somewhat problematic for children and elderly patients in the swallowing of the tablet. In all such patients, conventional solid formulations appear to be nonviable and poor patient compliance. The advantages of orodispersible tablets are enumerated as, it can be administered easily to patients having difficulty in swallowing like elderly, stroke victims, and pediatrics. This increases the bedridden patient's compliance, people travelling having less access to water. Orodispersible dosage form with good mouth feeling may help in strengthens the psychological belief on medication. Ease of administration to both young and elderly patients. More rapid absorption of drugs from pregastric parts of GIT improving the bioavailability and efficacy. Cost effective as minimum number of ingredients are required. Improved safety by prevention from the chocking or obstruction as in case of conventional dosage form during swallowing. The present dosage form provides medication in dissolved or dispersed form through solid dosage form.

Baclofen is rapidly and completely absorbed from the gastro-intestinal tract. A peak plasma level is generally reached within 0.5 to 1.5 hours and the plasma half-life is about 2 to about 6 hours and has an elimination half-life of about 3 to 4 hours.

Further, there are number of disadvantages are associated with liquid formulations and injectable formulation. In case of liquids, the main problem is to carry the bulky bottle and prevent it from breakage or accidently lost. Additionally, there are several other problems associated with liquid formulations are storage of the liquid dosage form, contamination, stability and accurate dosing. There are numbers of problems associated with injectable formulations like stability, aseptic feelings, free from accurate dosing, etc. Further, trained nursing staff or trained person required for administering the dosage. The side effects include skin rash, itching and pain.

U.S. Pat. No. 9,180,108 discloses a sterile injectable Baclofen formulation and method of manufacturing the same. The formulation is used in the implantable infusion devices. The formulation comprises baclofen in a concentration greater than 2 mg/mL, Sulfate or phosphate in a concentration of between 10 mM and 25 mM.

U.S. Pat. No. 10,610,502 discloses oral baclofen solutions. In one embodiment of the invention, the aqueous oral solutions comprise a buffer comprising citric acid, a salt of citric acid, or any combination thereof, and are stored at from about 2° C. to about 8° C. The present disclosure also relates to buffer free aqueous oral solutions comprising baclofen.

U.S. Pat. No. 11,324,696 discloses suspensions of metronidazole or baclofen and/or salts or ester derivative thereof. The suspension comprises metronidazole or baclofen, and/or a salt or ester derivative thereof a hydrocolloid stabilizer, simethicone emulsion, a buffer, such as sodium citrate, (dihydrate), a preservative, a thickening agent, a sweetener, and water.

There is still a necessity within society for pharmaceutical formulation of Baclofen that overcome all issues mentioned above and appropriate for Oral administration without any stability or dose uniformity issue. The present invention solves all prior arts problems and provide pharmaceutical composition for Oral administration comprising Baclofen.

### SUMMARY OF THE INVENTION

In accordance with present invention, the orodispersible solid pharmaceutical composition for Oral administration is prepared. The solid pharmaceutical composition comprising Baclofen, at least one disintegrant and at least one diluent.

In another embodiment of invention involves process for preparing the solid pharmaceutical composition for Oral administration. The granules prepared by using wet granulation process.

### OBJECTS OF THE INVENTION

The primary object of the present invention is to provide orodispersible a pharmaceutical composition of Baclofen.

Another object of the present invention is to prevent the dysphagia and improve patient compliance.

Still other object of the present invention is to provide a solid pharmaceutical composition of Baclofen suitable for Oral administration.

Still other object of the present invention is to provide process for preparing the solid pharmaceutical composition of Baclofen for Oral administration.

Yet Another object of the present invention is to provide stable and uniform oral dispersible pharmaceutical composition of the Baclofen.

Another object of the present invention is to provide fast disintegration to the dosage form as it gets in contact with saliva with good agreeable mouth feeling.

### DETAILED DESCRIPTION OF THE INVENTION

Solid pharmaceutical composition of Baclofen suitable for Oral administration is the invention as further described herein.

Further, the term “orodispersible”, used in the present invention, means that the tablets are uncoated tablets intended to be placed in the mouth where dispersed rapidly before being swallowed. The tablet disintegrates within 3 min after oral administration. The orodispersible tablets are also known as orally dispersible and orally disintegrating tablet.

The main embodiment of the invention is an orodispersible solid pharmaceutical composition suitable for Oral administration comprising Baclofen, at least one disintegrant and at least one diluent.

In a preferred embodiment, Baclofen present in the solid pharmaceutical composition as Baclofen. In one embodiment the Baclofen is present in the range from about 4% w/w to about 8% w/w, preferably in the range from about 5.5% w/w to about 7% w/w.

The term “about” as and where used in this specification means±10% of the mentioned value.

Orodispersible tablet (ODT) is a solid dosage form which disintegrates rapidly in the oral cavity after absorbing small amounts of saliva (1). Hence, it can be preferred by people having swallowing difficulty including children and elder. ODT relays on the presence of disintegrating agents (superdisintegrants) in the formulation. An appropriate disintegrating agent with appropriate percentage is a key aspect in the ODT dosage form development.

Drug bioavailability depends on drug absorption. In case of poorly water-soluble drugs, the bioavailability of the drug depends mainly on its dissolution that affected by drug dosage form disintegration. In case of low permeable drugs, it increases the absorption window by fast disintegrating the drug and providing larger surface area for absorption. Therefore, ODT formulation could help enhance the dissolution & absorption, then improving the bioavailability of these drugs. In addition, it can bypass the liver metabolism, when absorbed via oral mucosa.

In addition, Baclofen has an unpleasant taste and due to that poor patient compliance. Thus, the unpleasant taste of Baclofen needs to be masked in order to reduce poor patient compliance occurring when the active ingredient contacts the mucous membrane epithelium of the mouth.

The Solid pharmaceutical composition for Oral administration of the present invention is characterized by physicochemical properties suitable for a tablet formulation prepared by wet granulation, by adequate release rate of the active ingredient and storage stability achieved by employing excipients practically devoiding the tendency to interact with the active ingredient, and possessing good compressibility properties. The excipients were chosen carefully to give appropriate dissolution rate and stability of the finished dosage form. The ultimate goal was to develop a stable orodispersible formulation characterized by good taste and rapid disintegration which leads to greater absorption and high levels of the active ingredient in the systemic circulation.

As per one embodiment, the solid pharmaceutical composition of the present invention comprises Baclofen at least one disintegrant and at least one diluent.

In one embodiment, suitable diluent for present invention can be selected from microcrystalline cellulose, dextrates, dextrose, fructose, mannitol, Sorbitol, starch, pregelatinized starch, Sucrose, Xylitol, maltose, maltodextrin, maltitol and combinations thereof. In the present invention, combination of Microcrystalline cellulose and mannitol are used as diluent. Mannitol is used as diluent because of its negative heat of solution, sweetness, and ‘mouth feel’. Therefore, it is used in combination with Microcrystalline cellulose than over other diluent in the present invention.

In one embodiment diluent is present in the range from about 30% w/w to about 99% w/w, preferably from about 60% w/w to about 90% w/w.

In one embodiment, suitable disintegrant for present invention is selected from alginic acid, carbon dioxide, carboxymethylcellulose calcium, carboxymethylcellulose Sodium, croscarmellose sodium, guar gum, methylcellulose, polacrilin potassium, poloxamer, Sodium alginate and sodium starch glycolate. Further the disintegrant can be single or any combination of. Sodium starch glycolate is preferred disintegrant for the present invention present in the range from about 1% w/w to about 10% w/w, preferably in the range from about 2% w/w to about 7.5% w/w. Sodium starch glycolate, a representative example of a cross-linked starch, is a modified Starch possessing very significant disintegrating properties, and is practically insoluble in organic solvents. Sodium starch glycolate presents very good hydration capacity and very good flow properties in comparison to other Super disintegrants. Further, it presents the tendency to absorb water rapidly, so it swells in a significant amount. Therefore, this rapid water absorption by sodium starch glycolate molecules has as a result a significant increase in the Volume of granules resulting to rapid and uniform disintegration. Sodium starch glycolate incorporated in a pharmaceutical composition facilitates the breakup or dis integration of the content of the tablet into smaller particles that dissolve more rapidly than in the absence of disintegrating agents. Therefore, sodium starch glycolate is choice of disintegrant for orodispersible tablet.

As per another embodiment the solid pharmaceutical composition further comprises sweetener. The sweetener should be from about 0.5 to 10% w/w, preferably from about 1 to 6.5% w/w. In one embodiment, suitable sweetener for present invention is selected from acesulfame potassium, sucralose, cyclamate, saccharin, saccharin sodium and aspartame or mixtures thereof. In a preferred embodiment, Aspartame is to be used. The solid pharmaceutical composition of the present invention can be prepared in absence of sweetener as mannitol also act as sweetener.

As per one more embodiment, the solid pharmaceutical composition further comprising flavouring agent. Flavouring agents may be, for example, mint powder, menthol, orange flavour, Vanillin, aspartame or ace Sulfame potassium.

In one embodiment, suitable binder for present invention can be selected from the group consisting alginic acid, carbomer, ethyl cellulose, gelatine, glucose, guar gum, hydroxy ethyl cellulose, methylcellulose, polydextrose, polyethylene oxide and Povidone K30. Preferably Low substituted hydroxypropyl cellulose is preferred as a Binder for the present invention present in the range from about 0.25% w/w to about 7% w/w, preferably in the range from about 0.5% w/w to about 4% w/w.

Further as one embodiment the solid pharmaceutical composition of present invention, Lubricant is selected from boric acid, sodium benzoate, sodium olete, sodium acetate, sodium lauryl sulphate, magnesium stearate, sodium stearate, calcium stearate, steric acid, waxes or mixtures thereof. Magnesium stearate is preferred as a lubricant for the present invention present in the range from about 0.05% w/w to about 5% w/w, preferably in the range from about 0.2% w/w to about 2% w/w.

Thus as per one embodiment, the solid pharmaceutical composition of present invention remains stable at different temperature conditions.

One more embodiment of the present invention is to provide an orodispersible solid pharmaceutical composition suitable for Oral administration comprising Baclofen, at least one disintegrant and at least one diluent. The solid pharmaceutical composition further comprising at least one pharmaceutically acceptable excipient selected from sweetener, flavouring agent, binder, sweetener and lubricant.

Another embodiment of the present invention is to use of the wet granulation process for the preparation of orodispersible dosage forms of the present invention containing Baclofen, which is one of the most economical methods. Wet granulation is used mainly to improve flow and compressibility of powders and to prevent segregation of the blend components. It is used to convert a powder mixture into granules having Suitable flow and cohesive properties for tabletting. The wet granulation process is preferred to other common manufacturing processes because it improves the hardness of the tablets by reducing friability.

In the preferred embodiment, the disintegrating time of Baclofen orodispersible tablet is less than 3 minutes preferably less than 1 minute.

In the preferred embodiment, the wet granulation process comprising: Step 1: Weighing all raw materials individually as per the batch formula.

Step 2: Sieving mannitol, Microcrystalline cellulose 102, sodium starch glycolate, separately through #40 sieve. Aspartame, orange flavor, and magnesium stearate, separately through #60 sieve.

Step 3: Preparing binder solution by dissolving L-HPC in sufficient quantity of purified water and make a clear binder solution.

Step 4: Dry mixing of Baclofen, mannitol, and microcrystalline cellulose in the rapid mixer granulator.

Step 5: Adding binder solution gradually in dry mixed blend in rapid mixer granulator with continuance mixing.

Step 6: Drying the above granulated blend in a dryer at 50° C.±5° C.

Step 7: Passing dry granules through #24 sieve and retained granules milled through multi-mill and till all granules pass through #24 sieve.

Step 8: Mixing of granules with previously shifted Mannitol, sodium starch glycolate, aspartame, orange flavour in the blender.

Step 9: Mixing of the blend prepared in step 8 with magnesium stearate in a blender.

Step 10: Compressing the resulted mixture into tablet dosage form.

As per one more embodiment of the present invention the solid pharmaceutical composition of Baclofen is to be used for used in the treatment of spasticity resulting from multiple sclerosis, particularly for the relief of flexor spasms and concomitant pain, clonus, and muscular rigidity. It is also being of some value in patients with spinal cord injuries and other spinal cord diseases.

The invention is further illustrated by the following examples, which are by no means intended to limit the scope of the invention but are given by way of illustration.

### Example 1

| Ingredients | % w/w |
|---|---|
| Baclofen | 6.25 |
| Microcrystalline Cellulose 102 | 18.75 |
| Mannitol | 64.06 |
| Low substituted hydroxypropyl | 1.88 |
| cellulose | |
| Sodium starch glycolate | 3.12 |
| Aspartame | 2.50 |
| Orange flavour | 1.25 |
| Magnesium Stearate | 2.19 |
| Total | 100.00 |

### Manufacturing Process:

Step 1: Weighing all raw materials individually as per the batch formula.

Step 2: Mixing of Baclofen with approximately 1/10 quantity of the mannitol. Sieve mixture through 40 #sieve. Sieving remaining mannitol, microcrystalline cellulose-102, Low substituted hydroxypropyl cellulose, sodium starch glycolate separately through #40 sieve and aspartame, orange flavor and magnesium stearate through 60 #.

Step 3: Mixing one half of the amount of mannitol, One half of the sodium starch glycolate, microcrystalline cellulose 102, aspartame, orange flavor.

Step 4: Mixing of one half of the Baclofen and mannitol. Mixing of other half of the mannitol to above mixture. Then mixing other half of the sodium starch glycolate, microcrystalline cellulose 102, aspartame, orange flavor was mix.

Step 5: blending of the previously sifted magnesium stearate.

### Observation:

[symbol] The physical parameters of the blend were found not satisfactory.

[symbol] Poor flow of blend was observed.

| Test Parameters of blend | Result |
|---|---|
| Bulk Density (gm/ml) | 0.527 |
| Tap Density (gm/ml) | 0.721 |
| Carr Index (%) | 26.91 (poor) |
| Hausner Ratio | 1.36 |

### Example 2

| Ingredients | % w/w |
|---|---|
| Baclofen | 6.25 |
| Mannitol | 26.56 |
| Microcrystalline Cellulose 102 | 18.75 |
| Low substituted hydroxypropyl | 1.88 |
| cellulose | |
| Water | Q.s. |
| Mannitol | 37.50 |
| Sodium starch glycolate | 3.12 |
| Aspartame | 2.50 |
| Orange flavour | 1.25 |
| Magnesium Stearate | 2.19 |
| Total | 100.00 |

### Manufacturing Process:

Step 1: Weighing all raw materials individually as per the batch formula.

Step 2: Sieving mannitol, Microcrystalline cellulose 102, sodium starch glycolate, separately through #40 sieve. Aspartame, orange flavor, and magnesium stearate, separately through #60 sieve.

Step 3: Preparing binder solution by dissolving Low substituted hydroxypropyl cellulose in sufficient quantity of purified water and make a clear binder solution.

Step 4: Dry mixing of Baclofen, mannitol, and microcrystalline cellulose in the rapid mixer granulator.

Step 5: Adding binder solution gradually in dry mixed blend in rapid mixer granulator with continuance mixing.

Step 6: Drying the above granulated blend in a dryer at 50° C.±5° C.

Step 7: Passing dry granules through #24 sieve and retained granules milled through multi-mill and till all granules pass through #24 sieve.

Step 8: Mixing of granules with previously shifted Mannitol, sodium starch glycolate, aspartame, orange flavour in the blender.

Step 9: Mixing of the blend prepared in step 8 with magnesium stearate in a blender.

Step 10: Compressing the resulted mixture into tablet dosage form.

### Observation:

The physical parameters of the tablets were found not satisfactory.
Disintegration time was more than 3 minutes

### Example 3

| Ingredients | % w/w |
|---|---|
| Baclofen | 6.25 |
| Mannitol | 25.31 |
| Microcrystalline Cellulose 102 | 18.75 |
| Low substituted hydroxypropyl | 1.87 |
| cellulose | |
| Water | Q.S. |
| Mannitol | 37.50 |
| Sodium starch glycolate | 4.38 |
| Aspartame | 2.50 |
| Orange flavour | 1.25 |
| Magnesium Stearate | 2.19 |
| Total | 100.00 |

### Manufacturing Process:

Step 1: Weighing all raw materials individually as per the batch formula.

Step 2: Sieving mannitol, Microcrystalline cellulose 102, sodium starch glycolate, separately through #40 sieve. Aspartame, orange flavor, and magnesium stearate, separately through #60 sieve.

Step 3: Preparing binder solution by dissolving Low substituted hydroxypropyl cellulose in sufficient quantity of purified water and make a clear binder solution.

Step 4: Dry mixing of Baclofen, mannitol, and microcrystalline cellulose in the rapid mixer granulator.

Step 5: Adding binder solution gradually in dry mixed blend in rapid mixer granulator with continuance mixing.

Step 6: Drying the above granulated blend in a dryer at 50° C.±5° C.

Step 7: Passing dry granules through #24 sieve and retained granules milled through multi-mill and till all granules pass through #24 sieve.

Step 8: Mixing of granules with previously shifted Mannitol, sodium starch glycolate, aspartame, orange flavour in the blender.

Step 9: Mixing of the blend prepared in step 8 with magnesium stearate in a blender.

### Observation:

The physical parameters of the tablets were found not satisfactory.
Lamination was observed during tablets compression.

### Example 4

| | | Quantity |
|---|---|---|
| Ingredients | % w/w | (mg/tablet) |
| Baclofen | 6.25 | 20.00 |
| Mannitol | 26.56 | 85.00 |
| Microcrystalline Cellulose 102 | 18.75 | 60.00 |
| Low substituted hydroxypropyl | 1.88 | 6.00 |
| cellulose | | |
| Water | Q.s. | Q.s. |
| Mannitol | 37.50 | 120.00 |
| Sodium starch glycolate | 4.37 | 14.00 |
| Aspartame | 2.50 | 8.00 |
| Orange flavour | 1.25 | 4.00 |
| Magnesium Stearate | 0.94 | 3.00 |
| Total | 100.00 | 320.00 |

### Manufacturing Process:

As per experiment 3. In 10 mg tablet of the Baclofen, weight of all the excipients and Baclofen are 50% from of weight of 20 mg tablet. The total weight of 10 mg tablet is 160 mg. The manufacturing process is same as experiment 3.

### Observation:

All the physical and chemical parameters of Baclofen orodispersible tablets were found to be satisfactory.
IPQC (In Process Quality Control) data of lubricated blend are tabulated below.

| Test Parameters of blend | Result |
|---|---|
| Bulk Density (gm/ml) | 0.444 |
| Tap Density (gm/ml) | 0.538 |
| Carr Index (%) | 17.50 (Fair) |
| Hausner Ratio | 1.21 |

### Example 5: The Development Batch were Subjected to Stability Study 40° C.±2° C./75% RH±5% RH for 1 Month

Results are tabulated below.

### Baclofen 10 mg Orodispersible Tablets

| | | | | Exp. 4 | |
|---|---|---|---|---|---|
| | | | | (After 1 | |
| | | Exp. 4 | | month at | |
| Test | Specification | (Initial) | | 40° C. ± 2° C.) | |
| Description | White to off-white coloured, | Complies | | Complies | |
| | round shaped, flat-face, bevel | | | | |
| | edge uncoated tablets with | | | | |
| | break-line on one side and | | | | |
| | plain on other side having | | | | |
| | approximately 8 mm | | | | |
| | diameter. | | | | |
| Average | 160 mg ± 7.5% | 160.1 | | 160.3 | |
| weight | (148.0 mg to 172.0 mg) | | | | |
| Hardness | 20-100N | 64N | | 62N | |
| Thickness | 3.0 ± 0.3 mm | 3.01 | mm | 3.09 | mm |
| | (2.7 mm to 3.3 mm) | | | | |
| Friability | NMT 1.0% | 0.15% | | 0.17% | |
| Disintegration | NMT 3 Minute | 38 | sec | 45 | sec |
| time | | | | | |
| Wetting Time | NMT 3 Minute | 1 | min | 1 | min |
| | | 32 | sec | 34 | sec |

| | | | |
|---|---|---|---|
| Assay | 95.0-105.0% of label claim. | 101.9 | 99.9 |
| Dissolution | NLT 75% (Q) labelled | 96.3 | 96.8 |
| | amount of Baclofen should be | | |
| | dissolve in 15 minutes. | | |

Related Substances

| | | | |
|---|---|---|---|
| Impurity A | NMT 2.0% | ND (not | 0.062 |
| | | detected) | |
| Individual | NMT 0.15% | 0.036 | 0.036 |
| unspecified | | | |
| impurity | | | |
| Total | NMT 2.5% | 0.036 | 0.098 |
| impurities | | | |

### Baclofen 20 mg Orodispersible Tablet

| | | | | Exp. 4 | |
|---|---|---|---|---|---|
| | | | | (After | |
| | | Exp. 4 | | month at | |
| Test | Specification | (Initial) | | 40° C. ± 2° C.) | |
| Description | White to off-white coloured, | Complies | | Complies | |
| | round shaped, flat-face, bevel | | | | |
| | edge uncoated tablets with | | | | |
| | break-line on one side and | | | | |
| | plain on other side having | | | | |
| | approximately 11 mm | | | | |
| | diameter. | | | | |
| Average | 320 mg ± 5% | 321.0 | | 322.5 | |
| weight | (304.0 mg to 336.0 mg) | | | | |
| Hardness | 35-115N | 71N | | 85N | |
| Thickness | 2.8 ± 0.3 mm | 2.82 | mm | 2.89 | mm |
| | (2.5 mm to 3.1 mm) | | | | |
| Friability | NMT 1.0% | 0.19% | | 0.21% | |
| Disintegration | NMT 3 Minute | 43 | sec | 46 | sec |
| time | | | | | |
| Wetting time | NMT 3 Minute | 1 | min | 1 | min |
| | | 55 | sec | 40 | sec |

| | | | |
|---|---|---|---|
| Assay | 95.0-105.0% of label claim. | 97.3 | 99.6 |
| Dissolution | NLT 75% (Q) labelled | 92.2 | 96.7 |
| | amount of Baclofen should | | |
| | be dissolve in 15 minutes. | | |

Related Substances

| | | | |
|---|---|---|---|
| Impurity A | NMT 2.0% | ND | 0.049 |
| Individual | NMT 0.15% | 0.037 | 0.033 |
| unspecified | | | |
| impurity | | | |
| Total | NMT 2.5% | 0.037 | 0.083 |
| impurities | | | |

### Example 6: Stability Studies of Baclofen 10 mg

Stability study of composition of Example 4 was performed at 25° C.±2° C./60% RH±5% RH and 40° C.±2° C./75% RH±5% RH for 3 months. Results are tabulated below.

| | | | 3 M | |
|---|---|---|---|---|
| | | | 25° C./ | 40° C./ |
| Parameters | Specification | Initial | 60% RH | 75% RH |
| Description | White to off-white | Complies | Complies | Complies |
| | coloured, round | | | |
| | shaped, flat-face, | | | |
| | bevel edge uncoated | | | |
| | tablets with break- | | | |
| | line on one side and | | | |
| | plain on other side | | | |
| | having | | | |
| | approximately 8 mm | | | |
| | diameter. | | | |
| Hardness | 20-100N | 64N | 66 N | 58 N |
| Average | 160 mg ± 7.5% (148.0 mg | 160.1 | 160.2 | 160.5 |
| weight | to 172.0 mg) | | | |
| Disintegration | NMT 3 Minute | 38 sec | 42 sec | 48 sec |
| time | | | | |
| Dissolution | NLT 75% (Q) | 96.3 | 90.3 | 90.3 |
| | labelled amount of | | | |
| | Baclofen should be | | | |
| | dissolve in 15 | | | |
| | minutes. | | | |
| Assay (%) | 95.0-105.0% of | 101.9 | 100.5 | 102.1 |
| | label claim. | | | |

Impurities (%)

| | | | | |
|---|---|---|---|---|
| Impurity A | NMT 2.0% | ND | 0.2523 | 0.4342 |
| Individual | NMT 0.15% | 0.036 | 0.0458 | ND |
| unspecified | | | | |
| impurity | | | | |
| Total | NMT 2.5% | 0.036 | 0.2981 | 0.4342 |
| impurities | | | | |

Thus after 3 months' exposure to extreme temperature condition like 40° C., the solid pharmaceutical composition of Baclofen remains stable without any potency reduction or increase in impurity.

### Example 7: Stability Studies: Baclofen 20 mg

Stability study of composition of Example 7 was performed at 25° C.±2° C./60% RH±5% RH and 40° C.±2° C./75% RH±5% RH for 3 months. Results are tabulated below.

| | | | | 3 M | | | |
|---|---|---|---|---|---|---|---|
| | | | | 25° C./ | | 40° C./ | |
| Parameters | Specification | Initial | | 60% RH | | 75% RH | |
| Description | White to off-white | Complies | | Complies | | Complies | |
| | coloured, round shaped, | | | | | | |
| | flat-face, bevel edge | | | | | | |
| | uncoated tablets with | | | | | | |
| | break-line on one side | | | | | | |
| | and plain on other side | | | | | | |
| | having approximately | | | | | | |
| | 11 mm diameter. | | | | | | |
| Hardness | 35-115N | 71N | | 79N | | 77N | |
| Average | 320 mg ± 5% (304.0 mg | 321.0 | mg | 322.1 | mg | 321.7 | mg |
| weight | to 336.0 mg) | | | | | | |
| Disintegration | NMT 3 Minute | 43 | sec | 52 | sec | 48 | sec |

| | | | | |
|---|---|---|---|---|
| time | | | | |
| Dissolution | NLT 75% (Q) labelled | 92.2 | 91.7 | 91.5 |
| | amount of Baclofen | | | |
| | should be dissolve in 15 | | | |
| | minutes. | | | |
| Assay (%) | 95.0-105.0% of label | 97.3 | 103.3 | 101.7 |
| | claim. | | | |

Impurities (%)

| | | | | |
|---|---|---|---|---|
| Impurity A | NMT 2.0% | ND | 0.0412 | 0.0765 |
| Individual | NMT 0.15% | 0.037 | 0.0395 | 0.0397 |
| unspecified | | | | |
| impurity | | | | |
| Total | NMT 2.5% | 0.037 | 0.0807 | 0.1162 |
| impurities | | | | |

Thus after 3 months' exposure to extreme temperature condition like 40° C., the solid pharmaceutical composition of Baclofen remains stable without any potency reduction or increase in impurity.

1. An orodispersible pharmaceutical composition suitable for oral administration comprising Baclofen at least one disintegrant and at least one diluent.

2. The orodispersible pharmaceutical composition according to claim 1, wherein baclofen is present in the range from 4% w/w to about 8% w/w, preferably in the range from about 5.5% w/w to about 7% w/w.

3. The orodispersible pharmaceutical composition according to claim 1, wherein disintegrant is selected from alginic acid, carbon dioxide, carboxymethylcellulose calcium, carboxymethylcellulose Sodium, croscarmellose sodium, guar gum, methylcellulose, polacrilin potassium, poloxamer, Sodium alginate and sodium starch glycolate or combination thereof.

4. The orodispersible pharmaceutical composition according to claim 3, wherein disintegrant is sodium starch glycolate present in the range about in the range from about 1% w/w to about 10% w/w, preferably in the range from about 2% w/w to about 7.5% w/w.

5. The orodispersible pharmaceutical composition according to claim 1, wherein diluent is selected from microcrystalline cellulose, dextrates, dextrose, fructose, mannitol, Sorbitol, starch, pregelatinized starch, Sucrose, Xylitol, maltose, maltodextrin, maltitol or combinations thereof.

6. The orodispersible pharmaceutical composition according to claim 5, wherein diluent is combination of Microcrystalline cellulose and mannitol present in the range from about 30% w/w to about 99% w/w, preferably from about 60% w/w to about 90% w/w.

7. The orodispersible pharmaceutical composition according to claim 1, further comprising at least one pharmaceutically acceptable excipient selected from sweetener, flavouring agent, binder and lubricant.

8. The orodispersible pharmaceutical composition according to claim 7, further comprising Low substituted hydroxypropyl cellulose (L-HPC), Orange flavor, Aspartame and Magnesium stearate.

9. The orodispersible pharmaceutical composition according to claim 1 is for treatment of spasticity resulting from multiple sclerosis, particularly for the relief of flexor spasms and concomitant pain, clonus, muscular rigidity and in patients with spinal cord injuries and other spinal cord diseases.

10. A method for preparing an orodispersible pharmaceutical composition of Baclofen of claim 1, method comprising:
i. weighing all raw materials individually as per the batch formula;
ii. sieving mannitol, Microcrystalline cellulose 102, sodium starch glycolate, separately through #40 sieve and Aspartame, orange flavor, and magnesium stearate, separately through #60 sieve;
iii. preparing binder solution by dissolving L-HPC in sufficient quantity of purified water and make a clear binder solution;
iv. dry mixing of Baclofen, mannitol, and microcrystalline cellulose in the rapid mixer granulator;
v. adding binder solution gradually in dry mixed blend in rapid mixer granulator with continuance mixing;
vi. drying the granulated blend in a dryer at 50° C.±5° C.;
vii. passing dry granules through #24 sieve and retained granules milled through multi-mill and till all granules pass through #24 sieve;
viii. mixing of granules with previously shifted Mannitol, sodium starch glycolate, aspartame, orange flavour in the blender;
ix. mixing of the blend prepared in step (viii) with magnesium stearate in a blender; and
x. compressing the resulted mixture into tablet dosage form.

11. The method for preparing an orodispersible pharmaceutical composition of Baclofen of according to claim 10 is wet granulation method.

12. The orodispersible pharmaceutical composition according to claim 1, wherein the disintegrating time is less than 3 minutes preferably less than 1 minute.
